(19) United States
(12) Patent Application Publication
(10) Pub. No.: US 2025/0302452 A1
(43) Pub. Date: Oct. 2, 2025

(54) DEVICE FOR COLLECTING A BIOLOGICAL SAMPLE

(71) Applicant: PAVMED, INC., New York, NY (US)

(72) Inventors: Martin Von Dyck, New York, NY (US); Richard Yazbeck, Norwell, MA (US); Lishan Aklog, Purchase, NY (US); Michael Boutillette, Sudbury, MA (US)

(21) Appl. No.: 18/865,930

(22) PCT Filed: May 16, 2023
(86) PCT No.: PCT/US2023/022321

§ 371 Date: Nov. 14, 2024

Related U.S. Application Data
(60) Provisional application No. 63/342,209, filed on May 16, 2022.

(51) Int. Cl. A61B 10/00

(52) U.S. Cl. CPC A61B 10/0045; A61B 2010/0061

(57) ABSTRACT

A sample collection device, system, and methods, including a collection member designed to expand from a compressed configuration to an expanded configuration to contact a sample; a porous matrix imparted on the collection member and arranged in a gradient on the collection member, the gradient of the porous matrix having a range from about 10 pores per inch to 80 pores per inch; and a tether coupled to the collection member and extending proximally to permit pulling of the collection member in a proximal direction.

### CROSS-REFERENCE TO RELATED APPLICATION

The present application claims the benefit of priority to U.S. Provisional Application No. 63/342,209, filed May 16, 2022, and titled “DEVICE FOR COLLECTING A BIOLOGICAL SAMPLE.”

### BACKGROUND

The present disclosure is directed to a device for collecting a biological sample, and more specifically, to a device for collecting a biological sample, such as tissue, cells, protein, RNA and/or DNA from an esophagus of a patient.

A known tissue collection device includes an expandable device with longitudinally extending folds. The expandable device expands radially at a collection site within a body lumen, such as an esophagus. After the device is expanded, tissue is collected from the collection site. The expandable device is deflated after tissue is collected. The folds trap collected tissue when the device is deflated after collection of tissue. The known tissue collection device may be inserted through an endoscope to the collection site or via standard catheter intubation techniques. Such known tissue collection devices suffer from a number of drawbacks and complications.

### SUMMARY

The present disclosure relates to a sample collection device. The device includes a collection member designed to expand from a compressed configuration to an expanded configuration to contact a sample; a porous matrix imparted on the collection member and arranged in a gradient on the collection member, the gradient of the porous matrix having a range from about 10 pores per inch to about 80 pores per inch; and a tether coupled to the collection member and extending proximally to permit pulling of the collection member in a proximal direction.

In some embodiments, the collection member can have a semispherical shape in the expanded configuration. The collection member can have a diameter at its distal end that can be relatively larger than that along the remainder of the collection member. The collection member can be a size zero capsule, having a length of approximately 21.6 mm, when in the compressed configuration. The collection member can have a substantially spherical, cylindrical, pyramidal, truncated cone, bell, or button shape in the expanded configuration.

In some embodiments, the collection member can have a capsule shape in the compressed configuration. The gradient of the porous matrix can be configured in a plurality of separable layers. At least one of the plurality of separable layers can include pores capable of capillary action to draw a sample through the gradient of pores. The plurality of separable layers can include at least one hydrophilic layer. The plurality of separable layers can each have a distinct foam porosity value. The plurality of separable layers can have increasing foam porosity values from a proximal end of the collection member to a distal end of the collection member. The plurality of separable layers can include, a first, proximal, layer that can have a porosity of approximately 10 ppi, a second layer, being distal to the first layer, that can have a porosity of approximately 20 ppi, a third layer, being distal to the second layer, that can have a porosity of approximately 40 ppi, and a fourth layer, being distal to the third layer, that can be hydrophilic.

In some embodiments, the tether can extend through the first, second, third, and fourth layers to retain the layers together. The device can further include a capsule configured to maintain the collection member in the compressed configuration. The capsule can include a plurality of perforations to shorten the time for the capsule to dissolve or break down to release the collection member. In some embodiments, the perforations can be arranged in a row extending along a central axis of the collection member in the compressed configuration until the capsule is dissolved. In some embodiments, the perforations can be disposed in other arrangements, e.g., in a helical or spiral shape about the capsule. The plurality of perforations can be arranged in a plurality of rows and the plurality of rows are circumferentially offset from one another. The collection member can be impregnated with a binding material configured to maintain the collection member in the compressed configuration until the binding material is dissolved. The binding material can be one of hydromer polyurethane polyvinylpyrrolidone interpolymer, pure polyvinylpyrrolidone in water, or polyvinyl alcohol. The collection member may not include a capsule.

In some embodiments, thetether can be coated with a flavorant and/or a hydrophilic coating to aid in a patient's comfort during the insertion process. The tether can include a plurality of markings disposed along a length of the tether. The plurality of markings on the tether can be round suture bands. The tether can include a retainer disposed at a distal most end of the tether, the retainer can have a larger diameter than the tether and the retainer can be disposed against a distal most end of the collection member. The retainer can be glued to the tether. The retainer can be formed from a radiopaque material.

The present disclosure is directed to a system for collecting a biological sample in a patient. The system includes a collection member designed to expand from a compressed configuration to an expanded configuration at a site of interest to contact a sample; a matrix of pores imparted on the collection member, the matrix of pores having a gradient ranging from about 10 pores per inch to 80 pores per inch; and a pathway along which a fluid can be directed to the collection member to expand the collection member from the compressed configuration.

In some embodiments, the system can further include a capsule configured to maintain the collection member in the compressed configuration and the capsule being configured to dissolve upon interaction with the fluid. The capsule can include a plurality of perforations arranged in a row extending along a central axis of the collection member in the compressed configuration and areas of the capsule disposed between the plurality of perforations are configured to dissolve before the capsule is dissolved.

In some embodiments, the collection member can be impregnated with a binding material configured to maintain the collection member in the compressed configuration until the binding material is dissolved by the fluid. The binding material can be one of hydromer polyurethane polyvinylpyrrolidone interpolymer, pure polyvinylpyrrolidone in water, or polyvinyl alcohol. The collection member can, in some embodiments, not include a capsule.

In some embodiments, the collection member has a semispherical shape in the expanded configuration. A largest diameter of the semispherical shape can be arranged at a distal end of the collection member. The collection member can have a substantially spherical, cylindrical, pyramidal, truncated cone, bell, or button shape in the expanded configuration. The collection member can have a capsule shape in the compressed configuration.

In some embodiments, the system can further include an elongated tether having a distal end attached to the collection member and a proximal end to permit pulling of the collection member from the site of interest.

The present disclosure is directed towards a method for collection of a sample. The method includes, advancing, to a site of interest, a collection member in a compressed configuration, the collection member having (a) a matrix of pores arranged in a gradient having a range from about 10 pores per inch to 80 pores per inch, and (b) an elongated tether having a distal end attached to the collection member and a proximal end; moving the collection member against the site of interest to allow a sample to accumulate thereon; and pulling on the proximal end of a tether to remove the collection member from the site of interest.

In some embodiments, the method can further include directing a fluid through a pathway to the collection member such that the collection member expands from the compressed configuration to an expanded configuration to contact a sample. The collection member can include a capsule configured to maintain the collection member in the compressed configuration. The method can, in some embodiments, further include dissolving the capsule such that the collection member expands to an expanded configuration. The capsule can include a plurality of perforations arranged in a row extending along a central axis of the collection member in the compressed configuration, and wherein the method further includes dissolving material between the plurality of perforations to break apart the capsule. The collection member can be impregnated with a binding material configured to maintain the collection member in the compressed configuration until the binding material is dissolved by a fluid. The binding material can be one of hydromer polyurethane polyvinylpyrrolidone interpolymer, pure polyvinylpyrrolidone in water, or polyvinyl alcohol.

In some embodiments, the collection member can have a semispherical shape in an expanded configuration. A largest diameter of the semispherical shape can be arranged at a distal end of the collection member. The collection member can have, in some embodiments, a substantially spherical, cylindrical, pyramidal, truncated cone, bell, or button shape in an expanded configuration. The collection member can have a capsule shape in the compressed configuration.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1A is a side cross-sectional view of a collection member in a compressed configuration;

FIG. 1B is a side cross-sectional view of a collection member in an expanded configuration;

FIGS. 2A-2D are side views of alternative collection members according to the instant disclosure;

FIG. 3A is a side view of an exemplary multi-layer collection member;

FIG. 3B is a side view of an exemplary multi-layer collection member;

FIG. 4 is a side view of an alternative exemplary multi-layer collection member;

FIGS. 5A-5F show various shapes of exemplary collection members;

FIGS. 6A, 6B, and 6C show an exemplary capsule;

FIG. 6D shows an exemplary capsule;

FIG. 7 is a side view of an embodiment of a tether anchor; and

FIG. 8 is a side view of an embodiment of a tether.

### DETAILED DESCRIPTION

An illustrative embodiment of the present disclosure relates to systems and methods suitable for collection of a sample at a site of interest. Often, it can be desirable to advance a collection member into a lumen of a patient in a configuration that can be easily swallowable, or intubated, for the comfort of the patient. However, such a configuration does not provide suitable volume for collection of a sample at the desired site of interest. Therefore, the present disclosure provides for a collection member having a compressed configuration for advancement to the site of interest and an expanded configuration for collection of a sample. The instant disclosure additionally provides for collection members formed from porous materials having a porous matrix arranged in a gradient having a range from about 10 pores per inch to about 80 pores per inch. Arranging the porous matrix in a gradient allows for different sample types to be collected within different regions of the collection member. This advantageously allows for a single test to collect multiple sample types with a single collection member.

The present disclosure relates to systems and methods that can be used to collect then protect, or retain, collected cells from within a body. The system of the present disclosure can be used to advance a collection member to a target location within a body to be activated for cell collection. The collection member can be transformed between various states for different functions. For example, the collection member can be transformed from a compressed configuration to an expanded, collection, configuration. The compressed configuration can include transforming the collection member into a compact shape that can be suitable for navigation within a body lumen without getting caught, damaging, or otherwise interfering with the function of the body lumen. The expanded configuration can include modifying the size and/shape of the collection mechanism to enable at least one surface of the collection member to contact and sample materials from the body lumen. This state can include expanding a size and shape of the collection member.

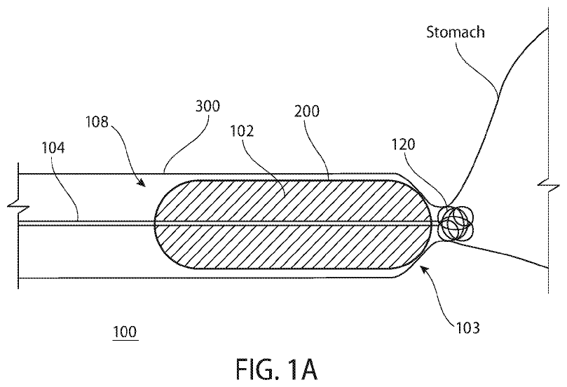

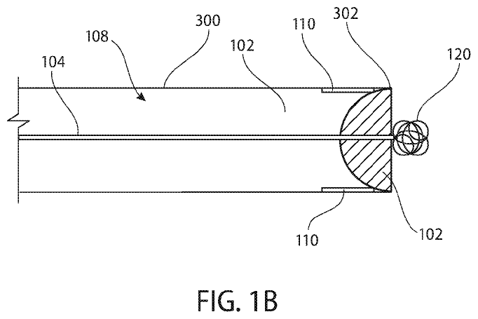

FIGS. 1A through 8, wherein like parts are designated by like reference numerals throughout, illustrate an example embodiment or embodiments of improved operation for sample collection, according to the present disclosure. Although the present disclosure will be described with reference to the example embodiment or embodiments illustrated in the figures, it should be understood that many alternative forms can embody the present disclosure. One of skill in the art will additionally appreciate different ways to alter the parameters of the embodiment(s) disclosed, such as the size, shape, or type of elements or materials, in a manner still in keeping with the spirit and scope of the present disclosure.

In general, FIGS. 1A and 1B illustrate a system 100 for collecting biological samples. System 100, in an embodiment, includes collection member 102 and a tether 104. In one embodiment, the collection member 102 can be made of a single material. For example, the collection member can be formed from any medical grade polymer foam material, e.g., reticulated polyether foam. The collection member 102 can be formed through a number of processes, each of which can generate a porous matrix gradient. For example, the porous matrix gradient can be a gradient of pore density within the collection member 102, such that the pore density, or porosity, increases or decreases across or along the collection member. In some embodiments, the porous matrix gradient can be a gradient of pore density extending from a proximal end to a distal end of the collection member 102. In an embodiment, the porous matrix gradient can be a gradient of pore density extending from a center point of the collection member extending outwardly.

In some embodiments, the porous matrix can be imparted with a gradient of pore size within the collection member 102. Meaning, pores with substantially similar diameters can be grouped such that there is a gradient of increasing or decreasing pore sizes, or diameters along or across the collection member 102. It should be noted that the range of pore diameters can vary and be dependent on the particular application. Further, in some embodiments, the pore size and the pore density can vary within a specific cross segment of the collection member.

In one example, the collection member 102 can be formed from a single piece of material that has a porous matrix arranged in a gradient within the single piece of material. For example, the collection member 102 can be 3D printed, or additively manufactured, to have the gradient imparted within the single piece of material. Alternatively, a subtractive manufacturing method, including laser drilling, may be used to laser drill the individual pores within the collection member 102. In the case of laser drilling, a foam collection member 102 may be formed with an initial geometry and then later laser drilled to obtain the desired porous matrix gradient. Additionally, or alternatively, the collection member 102 can be a reticulated foam which can be formed in a pressure chamber and then detonated, according to known methods. In such a reticulated foam, a laser drill can be used to open the cell faces to create the necessary foam structure.

The collection member 102 can have two general configurations during use. In a first, compressed, configuration, as seen in FIG. 1A, the collection member 102 can be collapsed or compressed in a capsule 103 having a generally pill shape. The capsule 103 can be any known size, for example a size zero capsule which can have a closed length of approximately 21.6 mm, with an approximate diameter of 7.64 mm-7.35 mm. In some embodiments, the capsule 103 can have any known standard size, for example 000, 00, 0, 1, 2, 3, 4, and 5. Such standard sizes can have standard dimensions as are known in the art. Generally, the smaller the capsule 103 the easier it can be for the collection member 102 to be inserted through a lumen, or body, 108 of a patient and advanced to a desired site of interest. However, the smaller the capsule 103 the more compressed the collection member 102 may need to be. It should be appreciated that if the collection member 102 is compressed beyond a certain size, its shelf life and effectiveness may be reduced as there can be a risk that the collection member 102 may not expand sufficiently. Therefore, the instant disclosure provides, among other things, in one embodiment, a semispherical collection member 102 which permits for a larger diameter collection member 102, as compared to a full sphere, while minimizing the total volume of mass that may be under pressure when in the compressed configuration. As such, the semispherical shaped collection member 102 can result in a more shelf stable collection member with a larger diameter, as compared to a full sphere-shaped collection member. The larger diameter semispherical collection member 102 can permit for a larger sample yield in an expanded configuration while additionally accounting for increased patient comfort in the collapsed configuration. In some embodiments, the larger diameter semispherical collection member can have a diameter of about 30-50 mm across its largest diameter. In particular, the diameter of the larger diameter semispherical collection member can be approximately between 30-35 mm.

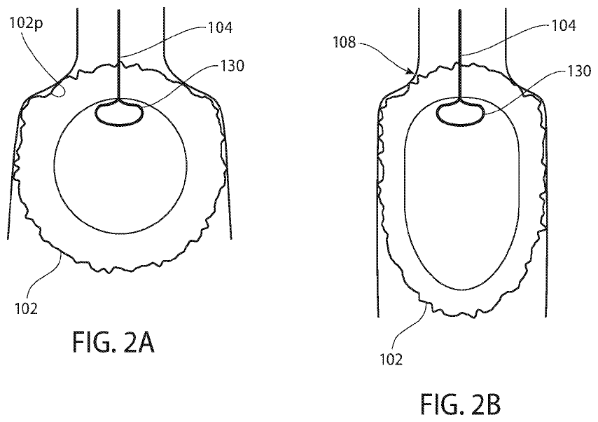

In addition, or alternatively, the collection member 102 can be hollow, as shown in FIGS. 2A, 2B, 2C, and 2D in order to minimize the total volume of mass under pressure. A hollow collection member 102 can, similar to the semispherical collection member 102 of FIGS. 1A and 1B, be compressed into a, relatively, smaller capsule shape while minimizing the total volume of mass that is under pressure when in the compressed configuration, similar to the capsule shape shown in FIG. 1A. The minimized total volume of mass of the collection member 102 that may be under pressure can provide for a more shelf stable product, as less mass is under pressure. Additionally, the hollow collection member 102 can have a shape, e.g., a sphere or a semisphere, which can allow for a constant force to be applied as the collection member is pulled through a lumen. Of note, because the collection member 102 of FIGS. 2A-2D are hollow, a spherical shape can be more easily achieved with the necessary dimensions to be swallowed in the collapsed configuration due to the reduction in overall mass. In an expanded configuration, the constant force applied by the hollow collection member 102 may allow for a more consistent sample collection. Further, a hollow collection member 102 can be designed to compress into different shapes, as a procedure requires, depending on where a tether may be retained in the collection member.

For example, as seen in FIGS. 2A and 2B, a tether 104 can be anchored on a proximal wall 102p of the collection member 102, with an anchor 130, or a series of knots 120 as is show in the embodiments of FIGS. 1A and 1B. In such an arrangement, the hollow collection member 102 can conform to the lumen 108, and form an elongated capsule shape, as seen in FIG. 2B, when the tether 104 is pulled in a proximal direction. The elongated capsule shape can allow for a larger contact area with the lumen 108 as the collection member 102 is being withdrawn.

Alternatively, as seen in FIGS. 2C and 2D, the tether 104 can be anchored on a distal surface 102d of the collection member 102. In such a configuration, the hollow collection member 102 can fold in on itself to protect a distal end 102d of the collection member 102, as the collection member 102 is being withdrawn through the lumen 108. Such a configuration can be advantageous if the distal end 102d of the collection member 102 is hydrophilic and a fluid from the site of interest is collected therein. In some embodiments, one or more layers of the collection member can have pores which are capable of capillary action to collect a sample. When the hollow collection member 102 is withdrawn from the site of interest, the distal end 102d of the collection member 102, now full of a sample fluid, will not abrade or otherwise contact the lumen 108 through which the collection member 102 is being withdrawn.

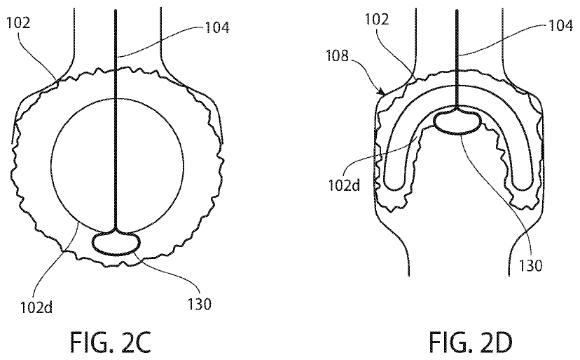

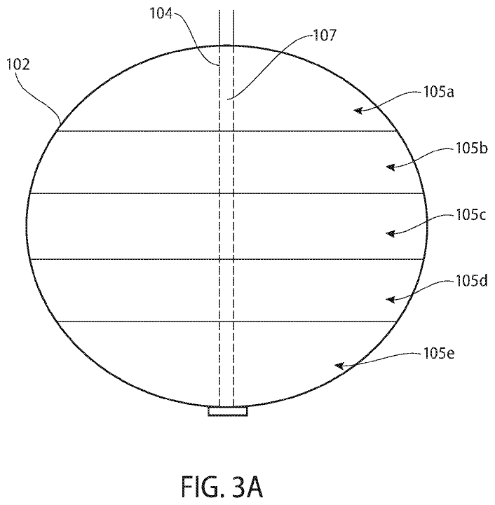

In an embodiment, the collection member can be formed from a plurality of distinct layers of material which can be stacked upon one another, as seen in FIG. 3A. The plurality of layers 105a, 105b, 105c, 105d, 105e can be stacked, one on top of the other, to form a collection member 102 having a desired shape. For example, as seen in FIG. 3A, the various layers can form a sphere. In some embodiments, each layer 105a-e can be formed from the same material, with each layer 105a-e having different porosities and durometers based upon the pores per inch present in each material layer. In other embodiments, the individual layers 105a-e can be formed of two or more different material types which can serve distinct purposes for the collection of a sample. These distinct layers of materials can have a constant porosity and durometer, or can have a gradient of porosities and durometers as well. Such a multi-layer collection member 102 can have at least two layers of material, at least three layers of material, or at least four layers of material. In one example, as seen in FIG. 3A, the collection member 102 can include five distinct layers of material 105a-e. Each layer of material 105a-e can have a distinct purpose and distinct material properties. The following discussion is merely one example of a combination of foams and is not intended to be a limiting example.

In some embodiments, a first, proximal most, layer 105a can form the top of a spherical shape and can be made of a foam having 10 ppi (pores per inch). The second layer 105b, which can be directly distal to the first layer 105a, can be made of a foam having 20 ppi. The third layer 105c, which can be directly distal to the second layer 105b, can be made of a foam having 40 ppi. The fourth layer 105d can be distal to the third layer 105c and can be made of a foam having 40 ppi. The fifth, distal most, layer 105e can be hydrophilic, or formed from a foam having 80 (or more) ppi, to absorb fluids for sampling. In one example, the first layer 105a can be used to collect H. pylori bacteria, the second through fourth layers 105b-d can be used to collect various parasitic organisms, and the fifth layer 105e can be used to collect a portion of a microbiome to make a diagnosis of other diseases. In some embodiments, the thickness of the respective layers can allow for the layers to remain flexible. In some embodiments, the layers can be the same or different thicknesses. Thus, the layered, or gradient, collection member 102 can have an advantage of collecting multiple types of samples all with a single collection member. This particular example of layers can additionally provide for a unique performance when being withdrawn from a lumen of a patient. For example, this collection member 102 can aid with the withdrawal of the collection member 102 by allowing the collection member to bow down when a withdrawal force is applied by the tether.

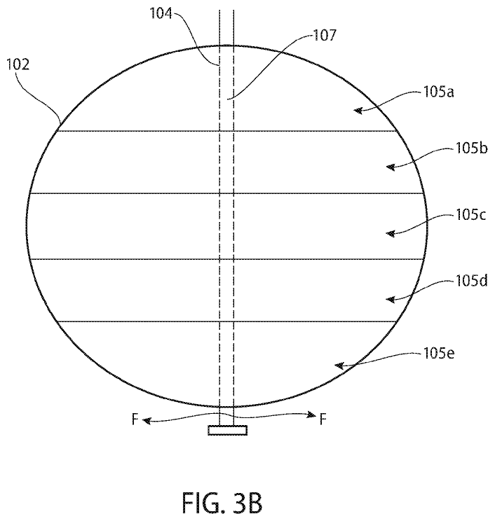

In another example, as shown in FIG. 4, the collection member 102 can be formed from three distinct layers 105a-c. For example, a first layer 105a of 20 ppi foam, a second layer 105b of10 ppi foam, and a third layer 105c of 20 ppi foam. The first and third layers 105a, 105c having 20 ppi foam can “sandwich” the second layer 105b of10 ppi foam such that the10 ppi foam may be in the middle. Once the layers 105a-c have been secured together via an adhesive, thermal bonding, or with a tether 104, the desired shape of the collection member can be cut. In some examples, the layers can be formed into a sheet of foam. The layers can have sufficient area to allow multiple collection members to be cut from the single sheet of material.

The various layers of the multilayer collection member 102 can be specially chosen to collect a variety of sample types, as a diagnostic test may require. The layers 105 themselves can be chosen on a test by test basis, or can be pre-chosen to detect specific ailments. The various layers 105 can be adhered together with an adhesive or with the use of thermal bonding Alternatively, the various layers 105 of a collection member 102 may not be adhered together and can be retained, axially, with a tether 104 only. In such a configuration, the radially outer edges of the layers 105 can move relative to the other layers to allow for the collection member to act akin to a “feather duster.” Such a “feather duster” collection member may provide for additional surface area contact between the collection member and the site of interest. Additionally, while a solid sphere is shown in FIGS. 3A and 4, it is contemplated that the collection member could be hollow or have other shapes, similar to the embodiments of FIGS. 2A-D.

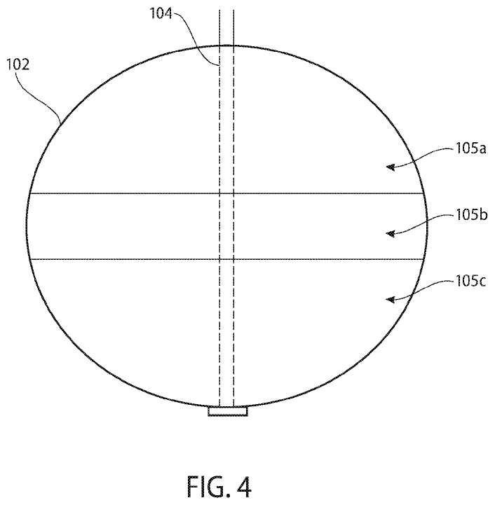

As shown in FIGS. 5A-F, the collection members can have a wide variety of different shapes. While solid geometries are shown, it is contemplated that any of the collection members disclosed herein can additionally be hollow. Further, in some examples, solid shapes are shown, however, such collection members 102 can include a plurality of layers, as discussed above with respect to FIGS. 3A and 4. In some examples, in addition to the semispherical shape of the expanded configuration of the collection member discussed above, the collection member can be shaped as one of spherical (FIG. 5A), cylindrical (FIG. 5B), pyramidal (FIG. 5C), truncated cone (FIG. 5D), bell (FIG. 5E), or button (FIG. 5F). In the case of the semispherical, pyramidal, truncated cone, or bell shapes, the collection member 102 can be oriented such that the diameter, or outer dimension, may be at a distal end of the collection member. The larger distal dimension can function as a net to aid in the collection of additional cellular material.

As noted above, the collection member 102 can have both a compressed configuration and an expanded configuration. The compressed configuration can be achieved via a number of different means which can retain the collection member 102 in shape that can be easy to deliver to a site of interest, while allowing for rapid expansion at a desired location. In one embodiment, as seen in FIG. 1A for example, the collection member 102 can be retained in the compressed configuration by means of a capsule 200. For example, the capsule 200 can be a gel cap made from gelatin or vegetable based dissolvable materials.

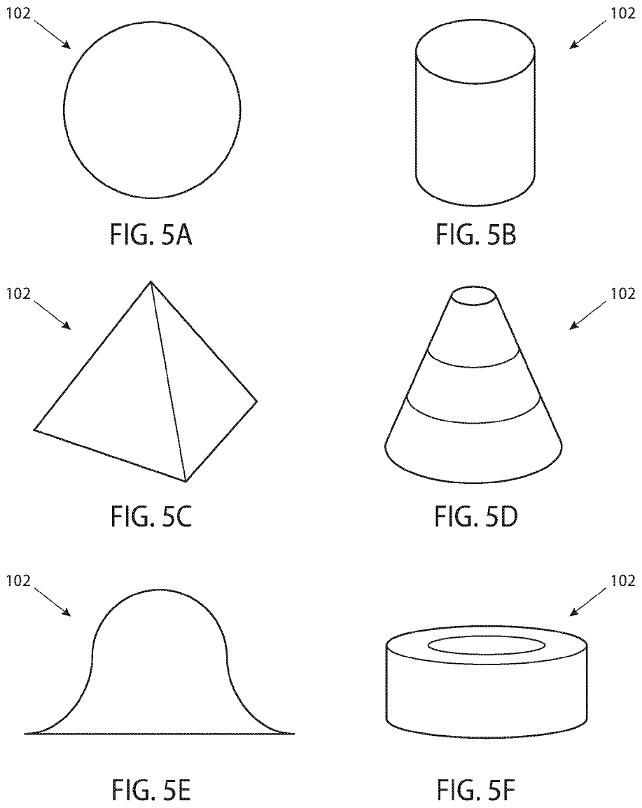

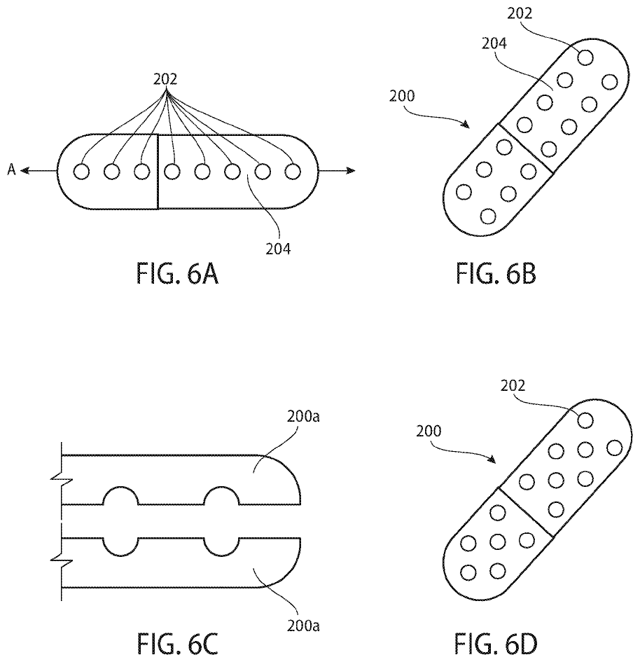

To facilitate the dissolving period, i.e., reduce the period of time over which the capsule can dissolve, capsule 200, as shown in FIGS. 6A and 6B, can be provided with pre-established “weak” points 204 along which the structural integrity of the capsule 200 can be compromised. This can be accomplished by a plurality of perforation 202 disposed about the capsule 200. In an embodiment, the perforations 202 can be arranged in pattern. For example, in one embodiment, the plurality of perforations 202 can be arranged in multiple rows of perforations, or holes, 202 extending along the length of the capsule 200. In one embodiment, four rows of perforations 202 can be aligned about the circumference and arranged every 90 degrees about a central axis A. Alternatively, more than four rows, or less than four rows, of perforations 202 can be included. However, the more rows of perforations that are included, the less stable the capsule 200 may be, due to the pressures of the collection member 102 in the compressed configuration pressing against the weakened walls of the capsule 200.

In one example, the perforations 202 can have a diameter of approximately 1.5 mm. These perforations 202 can serve two general functions. First, the weak points 204 can be defined as area of material between the perforations 202 in each row can be, relatively, minimized, such that it will dissolve quickly and act as a natural break point to open the capsule. Second, the perforations 202 can allow for a liquid to rapidly enter the capsule and cause the collection member 102 to swell and push open the capsule 200. In combination, the perforations 202 allow for the capsule 200 to break apart quickly, e.g., into parts 200a, 200b, in the presence of a liquid to rapidly split capsule with the compressed collection member, as can be seen in FIG. 6C. The liquid can be, for example, gastric fluid or other bodily fluids. Alternatively, or additionally, a patient can swallow water to introduce fluid to the sample site. In a further alternative, a practitioner can advance a catheter, or other tubing, to the site of interest and flush a fluid to the capsule with a syringe or other pump.

The perforated capsule 200 can be formed by a plurality of manufacturing processes. For example, the individual perforations 202 can be laser cut. Alternatively, a punch or “iron maiden” mold can be used to punch the perforations 202 into the capsule 200 itself. In a further alternative, the capsules 200 can be 3D printed with the perforations 202 formed in a wall. In yet another alternative, the capsule 200 can be molded to include the perforations 202 via the mold itself.

In an embodiment, as shown in FIG. 6D, the perforations 202 can be arranged in other geometric patterns. For example, the perforations 202 can be disposed in a “spiral” or a helical arrangement of perforations 202. In the embodiment of FIG. 6D, the capsule can rapidly split in the presence of liquid, similar to the embodiment of FIGS. 6A-6C.

In an alternative embodiment, the collection member 102 can be retained in the collapsed configuration without the use of a capsule all together. This can be achieved by impregnating the collection member 102 with a medically inert binding agent. For example, the collection member 102 can be soaked in a medically inert binding agent and then compressed into the desired compressed configuration, similar to the compressed configuration of FIG. 1A. Once the binding agent has dried, the collection member 102 can be retained in the compressed configuration by the now dried binding agent. Additionally, the compressed collection member 102 can be then be dyed and then packaged for use. In one example, the medically inert binding agent can be hydromer polyurethane polyvinylpyrrolidone interpolymer. When the hydromer polyurethane polyvinylpyrrolidone interpolymer is exposed to a fluid, e.g., gastric fluids or a warm solution swallowed by the patient, the material can fall apart, or dissolve, thereby allowing the collection member to expand to the expanded configuration as the binding material has been dissolved. In some embodiments, the hydromer polyurethane polyvinylpyrrolidone interpolymer can dissolve in approximately less than five minutes. In one embodiment, the dissolution time can be approximately three to five minutes.

Alternatively, the medically inert binding material can be pure polyvinylpyrrolidone in water. When pure polyvinylpyrrolidone in water is used as the binding agent, the collection member 102 can be soaked in the binding material and then compressed to the desired compressed shape. Once the collection member 102 is compressed, e.g., by a mold, the now compressed collection member 102 can be heated to drive the water out of the collection member 102, until the collection member 102 dries. The dry collection member 102 can then be dyed and packaged for use. In an embodiment, the binding agent can be polyvinyl alcohol. In an embodiment the medically inert binding material can be polyvinyl alcohol.

In some embodiments, the binding materials can include medications, in the form of a medicated binding agent, to target a disease. For example, the collection member 102 can be impregnated a medication that can be dissolved at a predetermined location within a patient. A pathway can be inserted in parallel with the collection member 102 such that a fluid can be injected directly onto the collection member 102 to dissolve the medicated binding agent to treat tissue at a desired location within the patient.

As discussed above, the collection member 102 can additionally include a tether 104 extending at least partially therethrough. The tether 104 can be used to retain the collection member 102 at a desired collection site and then used to remove the collection member 102 from a lumen 108 of the patient. In addition to this important feature, the tether 104 can additionally function to aid in the patient's comfort during the insertion process. For example, the tether 104 can be a round suture braid material. The round suture braid material can provide for a tether 104 with little, to no, elasticity and minimal sawing against tissue in the lumen 108. In an alternative, the tether 104 can be other types of sutures or an extruded materials. Alternative tethers 104 can have a certain amount of axial elasticity. For example, the tether 104 can stretch by 10% to effectively have shock absorbing characteristics, to prevent injury to a patient.

In the case of a round suture braid tether 104, it may be the case that the tether 104 is affixed to the collection member by means of a series of knots 120 tied at the distal end of the tether 104 to retain the tether within the collection member 102, as seen in FIGS. 1A and 1B. In some examples, the tether 104 can extend through a wall of a hollow collection member and a series of knots 120 can be tied such that the knots 120 can be disposed against an interior wall of the collection member, as shown in FIGS. 2A and 2B. The series of knots 120 can have a diameter that is larger than a through hole that the tether 104 extends through.

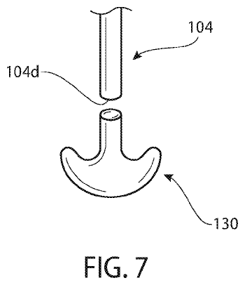

Alternatively, in place of the series of one or more knots 120, the tether 104 can have a molded retainer 130 at a distal most end 104d of the tether 104. The molded retainer 130, seen in FIG. 7, can be formed from any number of medically inert, body safe, materials. For example, the molded retainer 130 can be formed from a foam and then glued, or adhered, to the distal end 104d of the tether 104. Alternatively, instead of glue, the molded retainer 130 can be thermally bonded to the tether 104. In some examples, the tether 104 can be passed through the collection member 102 and then the molded retainer 130 can be adhered to the distal end 104d of the tether 104 on a distal side 102d of the collection member 102, or against an internal surface of the collection member 102.

In some embodiments, as seen in FIG. 7, the retainer 130 can have a generally anchor shape configured to conform to a generally spherical shape of a collection member 102. Alternatively, the retainer 130 can have a substantially disk, or button, shape with at least one through hole extending therethrough for connection to the tether 104. The retainer 130 can be formed from a foam material, for example any medical grade polymer material. In some examples, the retainer can be formed of a radiopaque material such as, but not limited to, barium sulfate (BaSO₄) and aromatic polyether-based thermoplastic polyurethanes (TPUs). Such radiopaque materials can permit for the physician to confirm that the retainer 130, and therefore the collection member 102, are properly positioned at the desired collection site during a procedure.

In some embodiments the tether 104 can have one or more coatings that can aid in patient comfort when the collection member 102 is swallowed, or otherwise inserted, into the esophagus of a patient. For example, the tether 104 can be coated with a flavorant which can make the sensation of swallowing the collection member 102 and tether 104 more palatable to the patient. Alternatively, or additionally, the tether 104 can be coated with a hydrophilic coating, e.g., HYDROMER. The hydrophilic coating can reduce the swallowing friction and tether sensation to the patient.

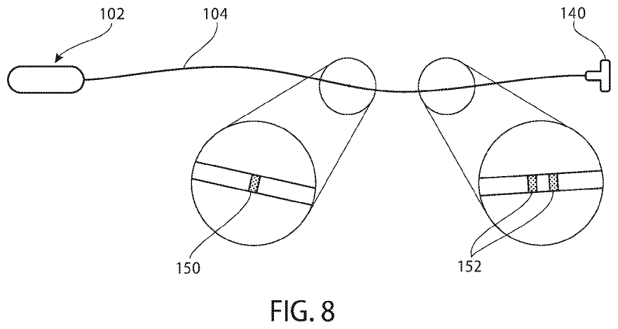

In some embodiments, as seen in FIG. 8, the tether 104 can, in general, include a handle 140 at a proximal end of the tether 104 and markings 150, 152 at predetermined intervals. The handle 140 can be used to apply a proximal withdrawal force to remove the system 100 from a patient. The markings 150, 152 can assist a physician with confirming the depth of the collection member 102 in the lumen 108 of the patient. For example, a first marking 150 can be at approximately 40 cm and a second marking 152 can be at 50 cm from the distal end of the tether. The 40 cm marking 150 can be a single marker band and the 50 cm marking 152 can be a double marker band. Alternatively, other markings can be located on the tether 104 and a guide, or legend, can be provided to the physician to indicate the predetermined depths.

In one exemplary method of use, the instant collection member 102 can be used to collect a sample 110 from a site of interest. For example, the collection member can be used to collect a biological sample of cells 110 from a lumen in a patient. In one exemplary embodiment, the lumen can be the upper digestive track, i.e., the esophagus 300, and the collection member 102 can be swallowed, or intubated, into the patient. The collection member 102 can be advanced through the esophagus until it has passed through the upper esophageal sphincter 302.

In operation, the system 100 of the present disclosure can be used for collection of materials within a body. Depending on the structure and configuration of the system 100, the collection member 102 can be transformable between the compressed configuration and the expanded configuration. The different states can modify the overall geometry of the collection member 102 and/or other components of the system 100 to be used for a particular purpose. For example, in the compressed configuration the size and/or shape of the collection member 102 can be generally capsule shaped for safe and comfortable navigation of the collection member 102 to a desired location within or out of the body 108. In another example, the expanded configuration can modify the size and/or shape of the collection member 102 for safely and comfortably using the collection member 102 to collect desired sampled materials at a particular location. The expanded configuration may protect any samples which may be acquired once the system 100 has been removed from the body, for example, to be analyzed/tested.

The system 100 can be used for cell, or sample, collection by first inserting the collection member 102 within a body 108 while in the compressed configuration. Insertion may be accomplished via swallowing, or intubation, of the collection member in the compressed configuration. The collection member 102 can be navigated within the body 108 to a point of interest that includes an area to be sampled. After the collection member 102 has been navigated to the target site for sample collection it can be activated. In some embodiments, bodily fluids may be used to activate the collection member 102 by dissolving one of a capsule 200 or a binding agent impregnated within the collection member 102. Additionally, or alternatively, in some embodiments, a patient may be instructed to swallow water or other fluid, at either room temperature or warmed to dissolve one of the capsule 200 or binding agents. Alternatively, a pathway, or catheter, can be inserted with the body 108 to direct a fluid at the collection member 102 to dissolve the capsule 200 or binding agent. The fluid can be injected through the pathway by an operator outside of the patient. In a further alternative, the capsule 200 can be dislodged from the collection member 102 with the use of pressurized gas, e.g., air, passed through the pathway. In some embodiments, the tether 104 can include, along its length, a lumen 107. Such a lumen 107 can provide the pathway for directing a fluid flow F to the collection member 102, as seen in FIG. 3B. While the collection member 102 of FIG. 3A is shown in FIG. 3B, it should be understood that a tether 104 with a lumen 107 can be used in combination with any collection member 102 disclosed herein.

In some embodiments, the collection member can include a capsule 200 that can be configured to maintain the collection member 102 in the compressed configuration for advancement to the site of interest. In embodiments where the collection member 102 includes a capsule 200, then it can be necessary to dissolve the capsule 200 to allow the collection member 102 to expand to the expanded configuration. In some embodiments, the capsule 200 can include a plurality of perforations 202 arranged in a plurality of rows extending along a central axis A of the collection member 102 in the compressed configuration. In such an embodiment, when the collection member 102 is exposed to a fluid, e.g., gastric fluid or other liquids, material 204 that extends between the plurality of perforations can dissolve to break apart the capsule 200 and thus allow the collection member 102 to expand to the expanded configuration.

In some embodiments, the collection member 102 may not include a capsule. In cases where the collection member 102 does not include a capsule 200, the collection member 102 can be impregnated with a binding material configured to maintain the collection member 102 in the compressed configuration until the binding material is dissolved by a fluid. In some embodiments, the binding material can be one of hydromer polyurethane polyvinylpyrrolidone interpolymer, pure polyvinylpyrrolidone in water, or polyvinyl alcohol.

The introduction of a bodily fluid, such as gastric fluid, or the introduced fluid can be used to initiate transformation of the collection member 102 between compressed configuration to the expanded configuration. Once the capsule 200 or binding agents is dissolved, the collection member 102 can freely expand to the expanded configuration. Once in the expanded configuration and the collection member 102 is at the cell collection site, the collection member 102 can be provided in contact with the body 108 to gather cells on at least one surface of the collection member 102. In some embodiments, the collection member 102 can initially collect gastric fluids with a hydrophilic layer at the distal end. The gastric fluid may have advantageous diagnostic benefits. As the collection member 102 is withdrawn from the stomach of a patient through the upper esophageal sphincter 302, the porous matrix of the collection member 102 can allows for collection of cellular material 110 for diagnosis. For example, the collection member 102 can have a porous matrix gradient that begins at10 ppi at a proximal end of the collection member and gradually increases to 20 ppi, then to 40 ppi, and up to 80 ppi. The portion of the collection member having the lower pores per inch, at the proximal end, can be considered to be more abrasive and will thus collect more material than the portion of the collection member having a higher pores per inch. For example, the proximal end of the collection member 102 can be used to collect H. pylori bacteria and as the porous matrix gradient transitions to a higher pores per inch, that distal region can collect various parasitic organisms, as seen in FIGS. 3A and 4.

Thereafter, the collection member 102 can be removed for retrieval and analysis of the collected materials (e.g., cells 110). In some embodiments of the collection member 102, the specific shape of the collection member can aid in the collection and protection of the collected materials 110, as the collection member 102 is removed from the patient. For example, in the case of a bell shaped (FIG. 5E), or semispherical shaped (FIG. 1B), collection member 102, the distal end of the collection member can have a relatively larger diameter, or dimension, which can act as a “net.” This distal net can function to collect a large amount of the collected materials due to the higher surface area.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present disclosure be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

1. A sample collection device, the device comprising, a collection member designed to expand from a compressed configuration to contact a sample;
a porous matrix imparted on the collection member and in a gradient, the gradient having a range of pore density from about 10 pores per inch to about 80 pores per inch; and
a tether coupled to the collection member and extending proximally to permit pulling of the collection member in a proximal direction.

2. The device of claim 1, wherein the collection member has a semispherical shape in the expanded configuration.

3. (canceled)

4. The device of claim 2, wherein the collection member has a capsule shape in the compressed configuration.

5.-7. (canceled)

8. The device of claim 1, wherein the gradient of the porous matrix is configured in a plurality of separable layers.

9. The device of claim 8, wherein at least one of the plurality of separable layers includes pores capable of capillary action to collect a sample.

10. The device of claim 89, wherein the plurality of separable layers includes at least one hydrophilic layer.

11.-17. (canceled)

18. The device of claim 1, wherein the collection member is impregnated with a binding material configured to maintain the collection member in the compressed configuration until the binding material is dissolved.

19. The device of claim 18, wherein the binding material is one of hydromer polyurethane polyvinylpyrrolidone interpolymer, pure polyvinylpyrrolidone in water, or polyvinyl alcohol.

20. The device of claim 18, wherein the collection member does not include a capsule.

21.-24. (canceled)

25. The device of claim 1, wherein the tether includes a retainer disposed at a distal most end of the tether, the retainer having a larger diameter than the tether and the retainer being disposed against a distal most end of the collection member.

26.-28. (canceled)

29. A system for collecting a biological sample in a patient, the system comprising:
a collection member designed to expand from a compressed configuration to at a site of interest to contact a sample;
a matrix of pores imparted on the collection member, the matrix of pores having a gradient of pore density ranging from about 10 pores per inch to about 80 pores per inch; and
a pathway along which a fluid can be directed to the collection member to expand the collection member from the compressed configuration.

30. The system of claim 29, further comprising
a capsule configured to maintain the collection member in the compressed configuration and the capsule being configured to dissolve upon interaction with the fluid.

31. The system of claim 30, wherein the capsule includes a plurality of perforations arranged in a row extending along a central axis of the collection member in the compressed configuration and areas of the capsule disposed between the plurality of perforations are configured to dissolve before the capsule is dissolved.

32. The system of claim 29, wherein the collection member is impregnated with a binding material configured to maintain the collection member in the compressed configuration until the binding material is dissolved by the fluid.

33.-38. (canceled)

39. The system of claim 29, further comprising an elongated tether having a distal end attached to the collection member and a proximal end to permit pulling of the collection member from the site of interest.

40. (canceled)

41. A method for collection of a sample, the method comprising,
advancing, to a site of interest, a collection member in a compressed configuration, the collection member having (a) a matrix of pores in a gradient of pore density having a range from about 10 pores per inch to about 80 pores per inch, and (b) an elongated tether having a distal end attached to the collection member and a proximal end;
moving the collection member against the site of interest to allow a sample to accumulate thereon; and
pulling on the proximal end of a tether to remove the collection member from the site of interest.

42. The method of claim 41, further comprising,
directing a fluid through a pathway to the collection member such that the collection member expands from the compressed configuration to an expanded configuration to contact a sample.

43. The method of claim 41, wherein the collection member includes a capsule configured to maintain the collection member in the compressed configuration.

44. The method of claim 43, further comprising dissolving the capsule such that the collection member expands to an expanded configuration.

45. The method of claim 44,
wherein the capsule includes a plurality of perforations arranged in a row extending along a central axis of the collection member in the compressed configuration, and
wherein the method further includes dissolving material between the plurality of perforations to break apart the capsule.

46.-52. (canceled)
